(19) United States
(12) Patent Application Publication
(10) Pub. No.: US 2011/0142959 A1
(43) Pub. Date: Jun. 16, 2011

(54) METHODS, SYSTEMS AND COMPOSITIONS INVOLVED IN THE SYNTHESIS OF NONSTABLE COMPOUNDS

(76) Inventors: William A. Farone, Irvine, CA (US); Tracy Palmer, Temecula, CA (US)

(21) Appl. No.: 12/966,081

(22) Filed: Dec. 13, 2010

Related U.S. Application Data
(60) Provisional application No. 61/286,274, filed on Dec. 14, 2009.

(51) Int. Cl. A61K 33/26; A61K 33/00

(52) U.S. Cl. 424/646; 424/722

(57) ABSTRACT

Systems, compositions, and methods for producing one or more unstable chemical species uniformly at an invariant rate of production over time and at substantially precise and/or exact concentrations are disclosed. The produced unstable product is synthesized from unstable chemical reactants. The present disclosure further describes methods of storage of an unstable chemical product to deliver it at a specific desired concentration at a specific time.

### CROSS REFERENCE TO RELATED APPLICATION

This application claims priority of U.S. Provisional Application No. 61/286,274 filed Dec. 14, 2009, the contents of which are incorporated fully by reference herein as part of this patent application.

### FIELD OF DISCLOSURE

The present disclosure relates to systems, compositions, and methods for producing one or more unstable chemical species uniformly, that is, at a substantially invariant rate of production over time and preferably at substantially precise and/or exact concentrations. The one or more chemical species may be synthesized from unstable chemical reactants. In a preferred embodiment, the unstable product may comprise a ferrate oxidant component which is produced in a chemical reaction involving an unstable chlorine oxidant compound. Very preferably, a composition of the present invention comprises a solution in which the ferrate oxidant is substantially or entirely free of residual hypochlorite, or more generally, in which the unstable product is produced in a composition substantially or entirely free of the unstable reactant. The present disclosure further relates to methods of storage of an unstable chemical product and, but not limited to, its use as a biocide.

### GENERAL BACKGROUND AND SUMMARY OF THE INVENTION

Unstable products produced from one or more unstable reactants in a chemical synthesis typically deteriorate at rate that varies with the concentration, phase of the material, solvent (if relevant), temperature, and time following production. In certain cases the unstable reactants can be stabilized by addition of a material that acts as a “negative catalyst” slowing the rate of the decomposition reaction but not substantially interfering with and/or preventing subsequent reactions. For example, in U.S. Pat. Nos. 6,790,429 and 7,476,324, and U.S. Patent Publication No. 2007/0217954A1 a portion of a metal ferrate solution is continuously generated in a reaction chamber located at or near the site of its desired bioremedial use, and delivered directly to the site following synthesis. A synthesis of stabilized mixed ferrates has been described in WO2001/28927 where stabilization is effected by partial substitution of Fe by a stable cation and by an excess of OH ions. Others have attempted to stabilize a metal ferrate solution by addition of sodium hypochlorite (CN101318707 A). In order to avoid the problems associated with the unstable metal ferrate oxidant produced from chlorine, others have used sulphate compounds (U.S. Pat. No. 5,746,994) for the production of a metal ferrate oxidant.

U.S. Pat. No. 6,974,562 discloses a continuous process, which involves a series of reaction chambers for producing a metal ferrate at the site of utilization. In the process disclosed in the '562 patent the concentration of the metal ferrate product at the site of use can vary from the concentration immediately as the metal ferrate is synthesized (the output), to a concentration that is greater than fifty percent of the said output concentration. The '562 patent does not disclose any way of controlling the output metal ferrate concentration.

The present invention relates to a system, method, and compositions involving the production of precise amounts of an unstable chemical product uniformly over time; in a preferred embodiment the unstable chemical product is a metal ferrate oxidant. In a particularly preferred embodiment the metal ferrate is made from a chemical reaction involving an unstable chemical reactant such as a soluble hypochlorite salt. The method of the present invention is particular useful for producing an unstable chemical product (or products) in a chemical reaction near or at a desired site at which they will be used (in situ). In additional preferred embodiments the invention is useful for metering the chemical products of such reaction(s) into another chemical process, or storage container. Examples of applications in which the present invention may be useful include: the production of metal ferrates (such as potassium or sodium ferrate) to be used directly as a biocide; the production of peracetic acid to be added directly in petroleum refining processes; and the generation of chlorine—hydrochloric acid leach solutions for ore processing.

A typical process of this invention involves and/or requires two or more reactants wherein at least one of the reactants is unstable. The unstable reactant will deteriorate at rate that varies with factors including concentration, temperature, phase, solvent and time. These reactants are stored at or near the site where they are to be used to produce the chemical product. The unstable reactants are stabilized by addition of a material that acts as a negative catalyst inhibiting the decomposition reaction but does not substantially interfere with subsequent reactions.

### BRIEF DESCRIPTION OF DRAWINGS

FIG. 1 Degradation of Ferrate (K₂FeO₄) in minutes at pH 10.7.

FIG. 2 Degradation of Ferrate (K₂FeO₄) in minutes at pH 11.8.

FIG. 3 Degradation of Ferrate (K₂FeO₄) in minutes at pH 11.2.

FIG. 4 Degradation of Ferrate (K₂FeO₄) in minutes at pH 10.4.

FIG. 5 Schematic Diagram of Process for Producing Unstable Chemical Species.

### DETAILED DESCRIPTION OF THE DISCLOSURE

The present invention is directed to systems, compositions, and related methods in which one or more unstable chemical reactants are utilized to produce one or more unstable chemical species, in precisely controlled amounts and concentrations, and at substantially uniform concentrations over a period of time. In a preferred embodiment reactants are mixed in separate mixing chambers that are each controlled reactors. The reactants are then added to each of two or more batch reactors in the proper stoichiometric ratio; the addition is sequentially accomplished; for example, one reactor is filled with the proper amounts of each reactant, then after a predetermined time period the next reactor is filled, and so forth, so that each chemical reaction is initiated at a sufficiently different time as to permit an essentially continuous supply flow of product at a predetermined flow rate, with the time staggered as to when each batch reaction is completed. Once one batch reaction has completed and the product has been completely delivered therefrom, a new reaction may be initiated in that reaction vessel so as to maintain the constant flow of product.

Preferably, the unstable product is produced in the reaction vessel at a concentration in the range of at least about 1% (w/v), or at least about 1.5% (w/v) or at least about 2% (w/v), or at least about 2.5% (w/v), or at least about 3% w/v) or at least about 3.5% (w/v) or more. In important embodiments of the invention, the unstable product, produced with no or substantially no unstable reactant, is stored after production diluted to concentrations of, for example, at least about 2000 mg/l, or at least about 2500 mg/l, or at least about 3000 mg/l or more. By “no or substantially no unstable reactant” is meant that no measurable amount of reactant is detected using spectrophotometric or ORP/pH-based monitoring methods. Storage of the product with monitoring of the storage tank permits the delivery of precise concentrations of the unstable product. Also, storage of the unstable product at such concentrations tends to stabilize the composition before delivery to its end use.

For example, when the unstable oxidant is sodium ferrate, the product can be delivered from the monitored storage vessel at a uniform concentration over time of at least about 8.5 mg/l. When potassium ferrate or another metal ferrate is used, the product may be delivered at a uniform concentration of at least about 10 mg/l. For example, the stored product may be diluted to these concentrations based upon the monitored storage concentration just prior at use. It will thus be understood that in this embodiment it is very important to monitor the stored product before delivery, since, if the delivered concentration were to fall below these values one could not be certain that the concentration would be sufficient to achieve its intended purpose.

Similar considerations apply for the synthesis and delivery of other unstable reactants and/or products.

In the preferred embodiment, the process described above requires at least two batch reactors. This mode of operation, known as a “fed batch” process, is not a continuous reaction process, but is capable of continuously providing a steady stream of product(s) from the reactions. For reactions that are based on one or more unstable reactant (reaction in which the quality and quantity of the produced chemical products may vary with temperature and/or the exact chemical composition of the input reactants) the fed batch mode has been found to be generally superior to the continuous reactor process since the reaction kinetics are not based on the input feed rate.

Furthermore, a fed batch operation allows one to know the concentration of one or more of the reactants and the concentration of one or more of the produced chemicals substantially at any instant before, during and after the reaction without reference to the previous storage system of the reactants once the reaction tanks are filled. After completion of the reaction the unstable product stream may be placed in a final product storage tank (for example, and without limitation, one for each reactor). The product is typically stabilized in this tank by the addition of a stabilizing chemical and typically delivered to its final use on a metered basis at a fixed concentration and rate.

In a preferred embodiment, at all, or essentially all, stages of the storage, reaction, and delivery process the composition of the unstable reactant(s) and or unstable product(s) are monitored to ascertain the concentration of the active ingredient(s). This information is then used to determine the timing for 1) the metering of the final unstable product to its final use, such as the metering of sodium or potassium ferrate into a cooling tower or other body of water to be treated), 2) the completion of the reactions in the fed batch reactors and 3) determining the initial concentration of the unstable reactants in the storage tanks. The monitoring can be carried out by any appropriate means, such as using spectrophotometric means (as disclosed in U.S. Pat. No. 5,262,961, incorporated by reference herein) or, in the case of chemicals with sufficiently different oxidation reduction potentials (ORP) between oxidized and reduced forms, by use of a combination of ORP and pH electrodes.

Monitoring a Reduction-Oxidation Pair

The general expression for monitoring a reduction oxidation pair is:

$$pE = log K - npH + log ( \frac{{[ Aox ]}^{m}}{{[ Ared ]}^{p}} ) \qquad [ 1 ]$$

The classic oxidation-reduction potential (ORP) used in an electrode meter is that ORP in millivolts is equal to 0.05915 times the pE. In equation [1] the terms given have the following meaning:

The expression p means the negative log of the concentration of some chemical entity, such as an ion, atom, electron, proton, charge or the like.

Thus, pH is the standard measure of acidity, given by equation [2].

pH=−log [H⁺] [2]

The pE is the analogous measure of the electron potential (or concentration), given by equation [3]

pE=−log [e⁻] [3]

The value n in Equation 1 equals the number of electrons transferred in the reaction being measured.

The K in the term log K (Equation 1) is the thermodynamic equilibrium constant for the reaction that is related to the change in Gibb's Energy going from m moles of the reduced form of the chemical to p moles of the oxidized form.

The expressions [Aₒₓ] and [A_{red}] are the oxidized and reduced concentrations (respectively) of an oxidizable chemical species in moles/liter.

For unstable compounds that are in a state of continuously changing, the measurement of ORP and pH is sufficient to identify the ratio between the oxidized and reduced forms, and thus track the concentration of the components according to equation [1]. This works as long as the only compound undergoing a redox reaction within a single reaction vessel is the compound of interest. In that case equation [1] can be rearranged and simplified to the form of equation [4], given below. Equation [4] indicates how the measurement of ORP and pH at various points in the process can define the concentration of the material changing form.

$$\frac{{[ Aox ]}^{m}}{{[ Ared ]}^{p}} = {10}^{( npH + ORP / 0.05915 - log K )} \qquad [ 4 ]$$

For the examples to be considered below this equation is a valid means of determining the concentration. In cases where the species of interest is not the only species in the system that is influenced by the pH and pE, a spectrophotometric method may be more convenient although equation [4] is valid for all oxidation-reduction pairs in a system simultaneously.

It will be understood that the monitoring methods described herein are preferred methods of monitoring, but that other methods of monitoring are available depending upon the chemical reaction to be monitored. These methods may indicated HPLC sampling of reaction and storage vessels through appropriate chromatographic media, such as ion exchange media, electrophoretic methods, and the like. The invention will therefore be understood not to be limited to any particular monitoring methods unless specifically so indicated.

A preferred process of the present invention, to be described further in the examples below, may be summarized as follows:

A method for accurately producing a desired concentration of an unstable chemical product from an unstable chemical reactant at a substantially uniform rate over time comprising the steps of:
a) separately storing two or more reactants, at least one of which is unstable;
b) monitoring the concentration of the unstable reactant;
c) placing the stored reactants into each of at least two reaction vessels at different times, thereby initiating chemical reactions in each storage tank at different times;
d) monitoring the concentration of at last one reactant and at least one product in each reaction vessel until a desired amount of an unstable product is produced free or substantially free of said unstable reactant.

The method may additionally or preferably involve transferring the unstable product from a reaction vessel into a storage or delivery vessel, and monitoring the concentration of the product continuously, or substantially continuously until the product reaches the site of use, so that the desired amount of the unstable product can be accurately delivered to the intended use.

Furthermore, the method preferably also further comprises adjusting the flow rate of the delivery stream from the two or more reaction vessels, to ensure substantially continuous delivery of the desired amount of said unstable product free or substantially free of said unstable reactant, and, when the reaction is complete in one reaction vessel, and the unstable product is delivered there from, the vessel is refilled with reactants and a new reaction is initiated, thereby permitting said continuous delivery to be substantially uninterrupted.

In a further embodiment, a composition containing a metal ferrate solution is produced by a reaction between ferric chloride, a chlorine containing oxidant and metallic hydroxide; and the ferrate solution is substantially free of residual hypochlorite. Preferably the metal ferrate is the sodium or potassium salt of ferrate. Preferably, the chlorine containing oxidant is a sodium or potassium hypochlorite salt in a solution comprising about 10% to about 13% metallic hydroxide; and a pH of at least 12. Preferably, the metallic hydroxide is selected from the group comprising sodium and potassium hydroxide.

In yet another embodiment, the invention is drawn to the method of synthesizing ferrate solution, and comprises:
a) monitoring the concentration of at least one unstable reactant;
b) Initiating a chemical reaction in a reaction vessel by combining reactants at least a chlorine oxidant, ferric chloride, and an alkali metal hydroxide under reaction conditions;
c) Removing a ferrate solution from the reaction vessel;
d) Recharging the reaction vessels with said reactants when necessary; and
producing substantially hypochlorite-free ferrate. The hypochlorite-free ferrate solution is then delivered to the desired site of use.

In another embodiment, the chlorine oxidant used to produce the ferrate solution is selected from the group consisting of sodium hypochlorite, potassium hypochlorite, and calcium hypochlorite. In an additional embodiment, the hydroxide used in the reaction is sodium hydroxide or potassium hydroxide.

In yet another embodiment a phosphate is added to the ferrate solution to stabilize the solution for storage and transportation. The phosphate is preferably selected from the group consisting of sodium phosphate and potassium phosphate. In a further embodiment the ferrate solution contains at least 0.05% phosphate. In a further embodiment then pH of the ferrate solution is maintained about 10 to 11.

In an additional embodiment the synthesis of the ferrate solution is conducted in a fed batch mode. The ferrate concentration and presence or absence of a chlorine oxidant is monitored in a further embodiment during the production of the solution. In an additional embodiment the ferrate solution concentration is monitored in storage to ensure that it remains active, for example, as a biocide.

In another embodiment, the remediation or biocidal treatment utilizes a ferrate solution containing at least 2000 mg/L of the ferrate measured as potassium ferrate or an equivalent amount of ferrate on a molar basis of any ferrate species in solution, preferably substantially in the absence of a hypochlorite component.

In certain embodiments the concentration of a ferrate composition has extended half life compared to otherwise identical ferrate compositions containing measureable amounts of hypochlorite.

The basic process will be exemplified with the production of sodium ferrate. For example, synthesis, according to the present invention, of potassium ferrate and peracetic acid would follow a similar method. Sodium ferrate is a known oxidant that decomposes in water. As an oxidant it is desirable because it can replace chlorine oxidants for applications where biocidal activity is required in various water treatments. The chlorine oxidants are well known to produce by-products (chlorinated organic compounds) that are toxic in very low concentration (5 micrograms per liter for most chlorinated acetic acid species). Sodium ferrate is itself unstable in water and decomposes to non-toxic by-products. Because of this decomposition, it is best used if it can be metered into water at a known concentration. In the case of the example to be given here, the selected delivery concentration is 10 mg/l; this amount is slightly greater (about 25% greater) than the concentration generally found to be effective for its use as a biocide.

A convenient reaction for producing these compounds uses a chlorine oxidant compounds such as sodium hypochlorite. The hypochlorite is also unstable. In a very preferred embodiment, there is no hypochlorite left after the ferrate itself is produced. Delivering a mixture of hypochlorite and ferrate negates the purpose of using ferrate in the first place, since the chlorine will contaminate the site. It is known that hypochlorites make trihalomethanes (THM) which are regulated as being toxic chemicals. Therefore the process of the present invention is designed to maintain and use the unstable sodium hypochlorite as a reactant, produce the unstable sodium ferrate in a form free of hypochlorite and maintain an output of 10 mg/l of ferrate.

As an example of an existing process for the production of ferrate, U.S. Pat. No. 6,974,562 discloses a device that operates on a continuous basis. This device consists of a series of chambers producing the ferrate mixture; this device produces at the site of use a concentration of ferrate equal to or greater than half the concentration of the ferrate at the output of the reaction chamber. The ferrate delivered to the site is not delivered at a fixed or precise concentration. There is no means disclosed in the '562 patent of insuring that any excess hypochlorite that is used is not delivered along with the ferrate, thereby contaminating the site. In all of the examples given in the U.S. Pat. No. 6,974,562 chlorine reactants are used and no provisions are made in the device to ensure that all the chlorine is utilized prior to discharging the final ferrate product. The continuous nature of the process creates changes in the concentration over time inevitable and yet no means is provided or disclosed of controlling or rendering constant the concentration of the product delivered.

U.S. Pat. No. 6,790,429 discloses essentially the same continuous device as in U.S. Pat. No. 6,974,562. Again in this case the oxidant used to make the ferrate is not totally used before the ferrate is discharged and therefore the other oxidant can also contaminate and be delivered along with the ferrate. The ferrate concentration as delivered is not controlled nor is it constant. Ciampi appears to provide an iron salt and an oxidizing agent in a mixing chamber mixed to provide a mixture without any reaction since it is claimed that at least a portion is delivered to a reaction chamber where it is reacted. It does not seem possible to continuously mix an iron salt and an oxidant in one chamber and to prevent a reaction from occurring until a portion of the mixture is placed in another chamber.

Other examples in the art likewise do not describe a method capable of insuring that no other oxidant enters the final treatment system, delivering a uniform amount of ferrate oxidant, delivering the ferrate at a desired use concentration that may be considerably lower than the concentration in the reaction tank.

Definitions

In accordance with the present disclosure and as used herein, the following terms are defined with the following meanings, unless explicitly stated otherwise.

The term “ferrate” refers to the anion [FeO₄]²⁻ in which iron is in the +6 formal oxidation state and is matched by cations of sodium or potassium ion.

The term “site” refers to any location where the reaction product may be stored or may be used.

The term “fed batch” refers to a batch process operation mode wherein at least two reactors are filled with reactants in the proper ratio sequentially such that a continuous supply of reaction products occurs.

The term “ORP” refers to the oxidation-reduction potential of a chemical reaction.

The term “THM” refers to trihalomethanes.

The term “negative catalyst” refers to an agent that reduces the rate of a reaction; and also refers to inhibitors of such reaction.

The term “half life” refers to the time for the concentration at the site to be reduced to one half of the initial concentration after the reaction.

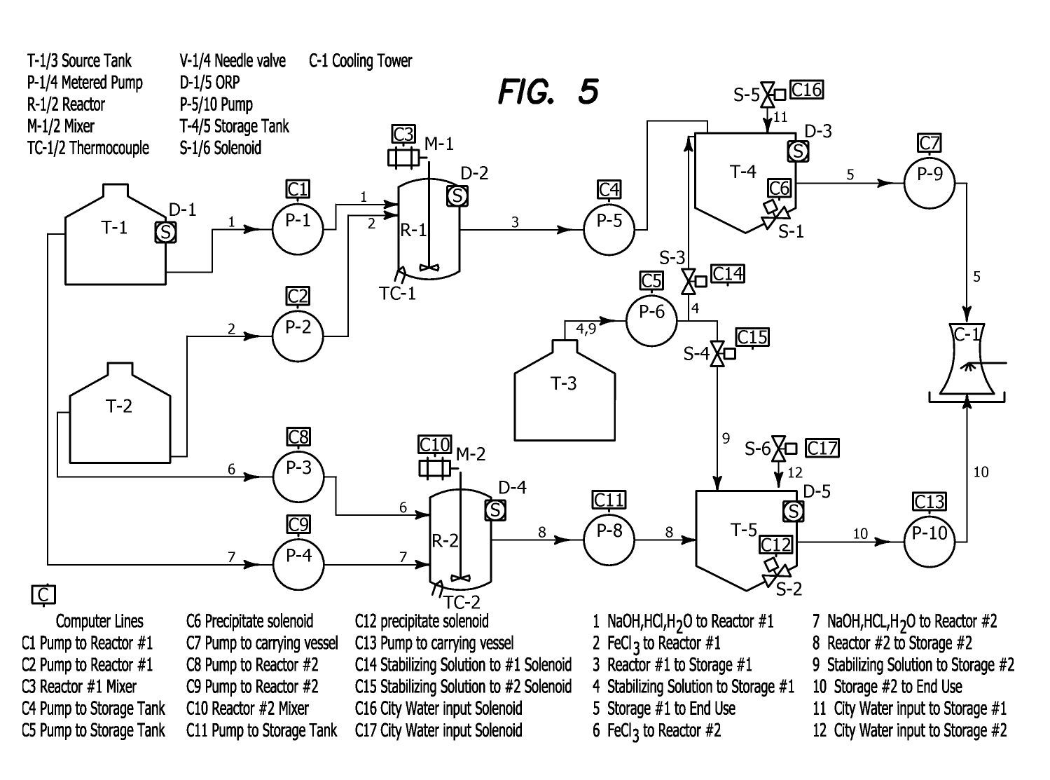

The use of this process for sodium ferrate production may be further understood by reference to FIG. 5. The general reaction for sodium ferrate is shown in equation [5].

2FeCl₃+3NaOCl+10NaOH=2Na₂FeO₄+9NaCl+5H₂O [5]

In this reaction the NaOCl is the unstable reactant and the Na₂FeO₄ is the unstable sodium ferrate product. The produced sodium ferrate breaks down according to equation [6].

4Na₂FeO₄+10H₂O=4Fe(OH)₃+8NaOH+3O₂ [6]

The NaOCl releases oxygen gas and HCl vapors which drives the otherwise very slow reactions in equation [7] and [8].

NaOCl═NaCl+½O₂ [7]

NaOCl+H₂O═NaOH+HOCl═NaOH+HCl+½O₂ [8]

Referring to FIG. 5, Tank T-1 contains a stored solution of NaOCl and NaOH. Tank T-2 contains a stored solution of FeCl₃. Generally in the art, the FeCl₃, NaOCl and NaOH are each added separately to the reaction and three tanks would be needed to store the reactants. It has been long felt by the providers of NaOCl that NaOH cannot be used to stabilize NaOCl. However, it was found by the present inventors that NaOH stabilizes the unstable NaOCl. Further stabilizing ingredients such as sodium phosphates may also be added to allow the combined reactants to be stored for an extended period of time until needed.

The notation of “S” in a box wherever it appears in FIG. 5 indicates a sensor to monitor the concentration of one or more of the reactants in that part of the system. As an example, the “S” in T-1 monitors the concentration of NaOCl to ensure it is in the correct concentration range for the subsequent reaction. As noted previously in this disclosure, the sensor can use any suitable monitoring methodology. Preferably the monitoring system may be a combination of pH and ORP, or a spectrophotometric sensor.

Upon demand the reactants are pumped into either R-1 or R-2 reaction vessels. When the reactants are pumped to a reaction vessel the reaction is allowed to occur. The ferrate is formed according to equation [6] and the concentration is monitored by the sensor (“S”) attached to the particular reactor.

In the specific case of ferrate the final product will eventually be dispensed to a use such as indicated by the label C-1 in the diagram (FIG. 5). For example, when where oxidization of cooling tower water is desired, if the inlet water flow to the cooling tower was 200 gallons per minute (gpm), and it is desired to keep the ferrate concentration in the tank at 10 mg/l, then the storage tanks T-4 and T-5 would have a concentration of at least 2000 mg/l of ferrate and the input solution would be delivered at about 1 gallon per minute to provide the necessary concentration.

Returning to the reaction in R-1, the ferrate produced in this example has a concentration significantly higher than that desired for the storage concentration. A useful target in this example is 2-3% w/v ferrate in the final reaction mixture; the concentration is monitored as indicated above. Importantly, at the completion of the reaction it is determined, again through monitoring, that there is no residual NaOCl in the mixture and there is at least 2% (for this example) ferrate. As an example, if the reactors R-1 and R-2 are chosen to have a volume of at least 5 gallons each, then 5 gallons of at least 20,000 mg/l ferrate is produced with no or substantially no other oxidant present.

Ferrate degrades according to equation [6]. It has also been found that this degradation is enhanced by the presence of Fe(OH)₃. Thus, as the Na₂FeO₄ degrades, the degradation speeds up. It is known in the art that this degradation can be retarded at very high NaOH concentrations. This is due to the effect in equation [6] of driving the “reverse” reactions due to large excess of NaOH on the right side of the equation as written. This fact is used, for example, to make ferrate electrolytically where Na₂FeO₄ is made in 18 Molar (720 grams/liter) NaOH solution and K₂FeO₄ is similarly made in 18 Molar (1010 grams/liter) KOH. However, these high concentrations of base are not useful in the case of delivering ferrate for many uses because 1) it would require an excessive amount of neutralization with other chemicals to keep from causing a large increase in the basicity in the application solution and 2) it would increase the cost of chemicals, prevention of deposition of scales and the cost of materials of construction to offset the deterioration caused by the high pH.

It has been found, however, that even though the degradation increases as the pH is lower than the above 14 in the extreme NaOH and KOH solution, that there is a point of product stability at about pH 10.4, particularly in the presence of phosphate. The phosphate not only stabilizes the ferrate by forming a stable peroxide, but it also maintains the pH at the desired level. A similar effect is described in part in U.S. Pat. No. 4,574,084 where it is disclosed the use of phosphates to stabilize metal peroxides.

The solution from R-1 after the reaction has completed is placed into T-4 and diluted with water and the stabilizing solution from T-3 to about 1000 mg/l or about 1.5 mg/l, or about 2000 mg/l or about 2500 mg/l or about 3000 mg/l. The stabilizing solution in T-3 is a combination of buffering and stabilizing agents; phosphate solutions in the case of ferrate. For example, when the solution is 5 gallons of at least 20,000 mg/l ferrate in R-1 it will be diluted 10:1 to make 50 gallons of at least 2,000 mg/l ferrate in T-4. Each gallon of solution in T-4 can then be used to treat 200 gallons of final treated water at 10 mg/l. Note that the sensor “S” in T-4 continues to monitor the concentration of ferrate in the tank. Based on the concentration and the desired flow to match the final use pump P-9 can be adjusted by computer control C-7 to precisely set the amount of ferrate being utilized for the treatment. This is also an important aspect of this embodiment of the invention.

The above-described dual “batch fed” system allows for the precise and constant amount of unstable product to be delivered as needed to the site of use without fear of running out of ferrate solution. While T-4 is delivering ferrate to the end use, T-5 is being filled from reactor R-2 with a properly timed reaction. Each of the pumps P-1 to P-10 is controlled by a corresponding computer control switch. There are also controls on the buffer-stabilizer tank T-3 for the two solenoid valves S-3 and S-4 to alternate delivery of the stabilizing and buffer solution. Solenoids S-1 and S-2 allow the tanks to be drained if necessary in those cases where the system is stopped before the tanks are emptied. Solenoids S-5 and S-6 add the rest of the dilution water to T-4 and T-5 respectively after the solutions are added from R-1/T-3 or R-2/T-3 respectively. This is controlled by level sensors in T-4 and T-5.

This system is very general and can include many reactions of unstable chemicals for which delivery of a precise amount is required. Another example utilizing the process as described above, there is a need for either performic acid or peracetic acid as an oxidant for organic chemical reactions. For example, in the conversion of oleic acid to 9,10-dihydroxystearic acid, it is desirable to deliver one of these oxidants to a continuous reactor which can also be represented by C-1 of the diagram. For this example the reaction in R-1 and R-2 is given by equation [9] written for performic acid.

H₂O₂+H(CO)OH=H(CO)OOH+H₂O [9]

In this case the hydrogen peroxide is the unstable reactant. The hydrogen peroxide can be stabilized with various phosphorus compounds and stored in T-1 as previously described above. The reactions in R-1 and R-2 are monitored for the formation of the desired product and the resulting unstable product is also monitored and stored in T-4 and T-5 and dispensed as needed. The reaction with acetic acid to make peracetic acid would proceed in a similar manner.

A further example is the formation of a solution that is rich in chlorine which can be utilized for the leaching of ores. In this example C-1 is an enclosed conveyor that has an ore which contains gold or platinum group metals and the ore is carried via the conveyor. By way of explanation the leached solution will be treated to inactivate the chlorine in the next stage with another reagent. However, the focus is simply on the part of the process that uses the currently disclosed method.

The reaction for the leaching solution is given in equation [10].

HCl+NaOCl═Cl₂+NaOH [10]

The reaction in equation [10] is carried out in the presence of excess HCl with no formation of NaOH. The reaction shown in equation [11] gives the details of the reaction in this example where NaOH is not formed.

2HCl+NaOCl═Cl₂+NaCl+H₂O [11]

The reaction is carried out in aqueous solution wherein the reactants and products are soluble.

In the above example, NaOCl is again the unstable starting material and the produced Cl₂ is the unstable product. It was found that the addition of NaOH still be used as a stabilizer for the NaOCl and this addition simply requires more HCl for the reaction. The adjustment of pH for the finished product is critical for the use of the product in leaching ores with an approximate pH of 2 being preferred. A process embodying the use of such a solution to leach ores is given in U.S. Pat. No. 6,551,378.

A very large number of additional applications of the method are possible. The following examples provide further information on various elements of the method enumerated above.

### EXAMPLES

### Example 1

### Degradation of Ferrate

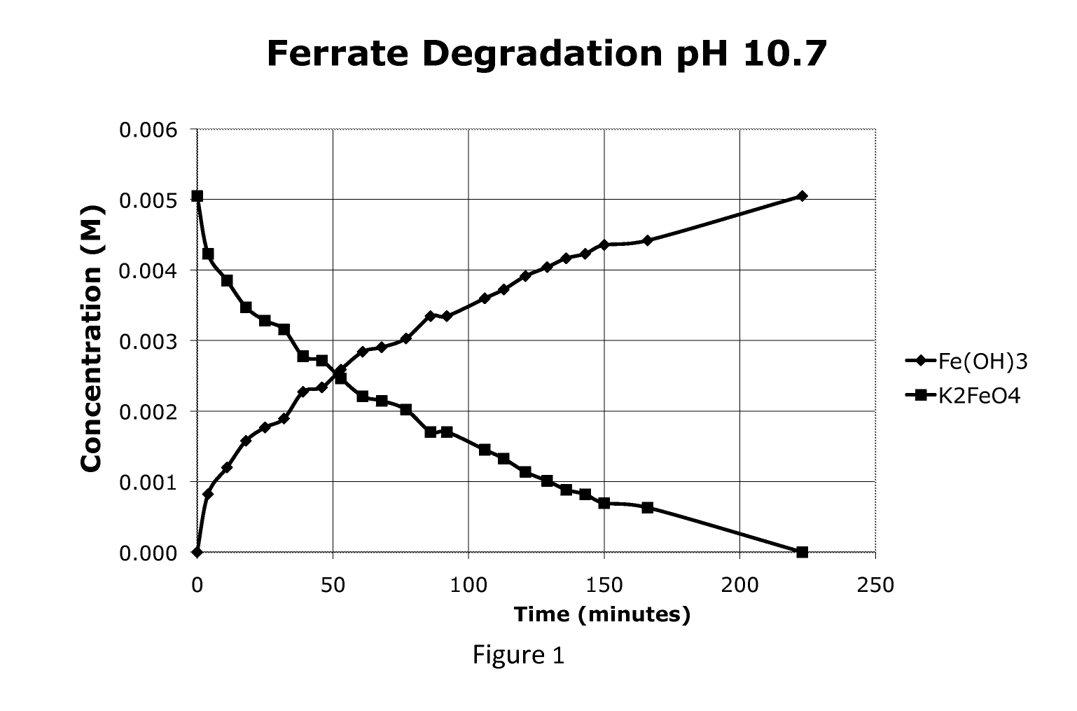

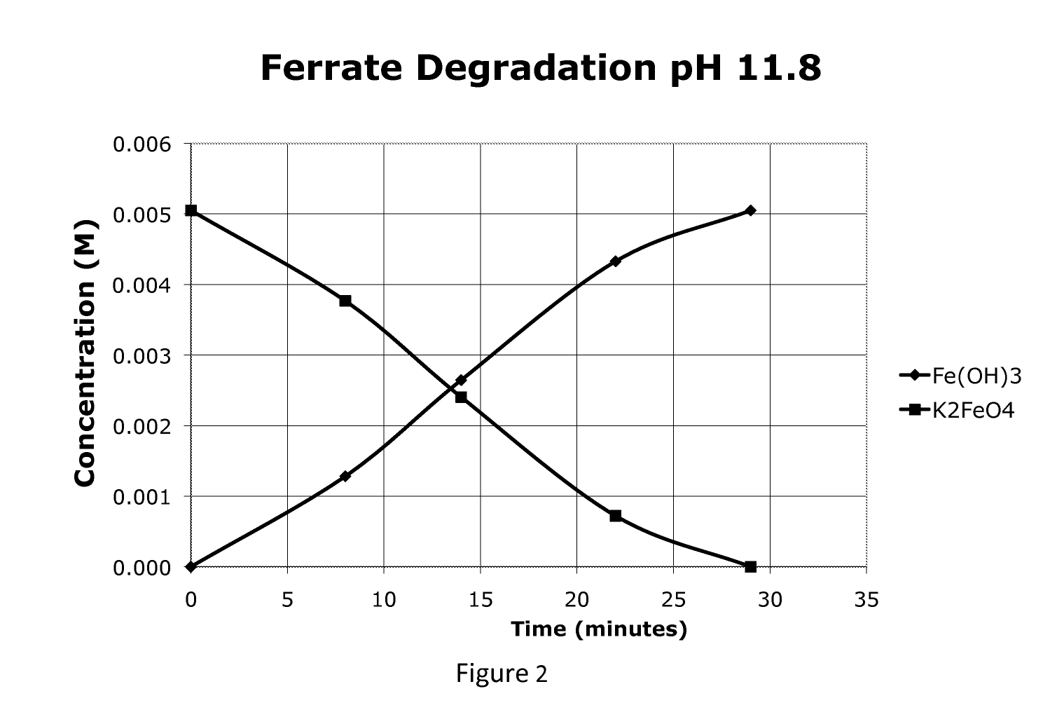

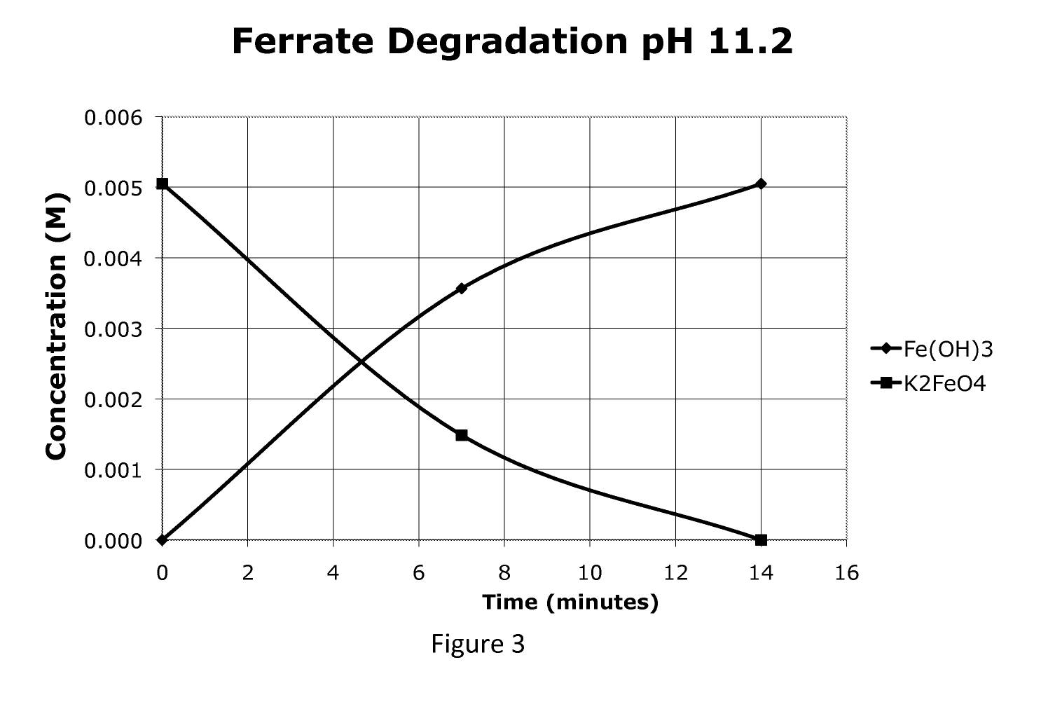

FIG. 1 is a profile of the ferrate concentration starting at 0.1% (5 mM) at the native pH of K₂FeO₄, about 10.7. As seen in FIG. 1 the ferrate was present in the solution for about 220 minutes. FIGS. 2 and 3 show the ferrate degrading faster at the higher pH's recommended by the Ciampi et al patent(s). Within about 21-46 minutes in the range of pH 11.2-11.8 the ferrate completely degraded. At this higher pH the Fe(OH)₃ forms much faster and contributes to the acceleration in degradation.

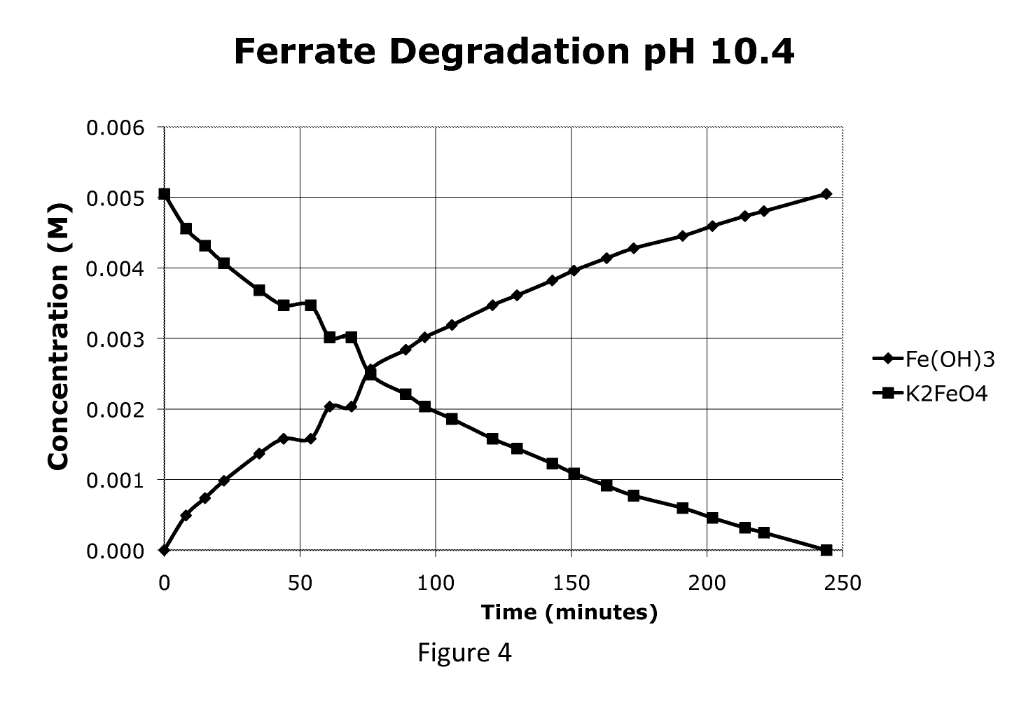

FIG. 4 discloses a slightly lower pH of 10.4 which was buffered to approximately pH 9 with NaOH. Ferrate was detected in the solution for about four hours.

### Example 2

### Preparation of Sodium Ferrate

Sodium ferrate was produced as follows:

A solution which contained 164 grams of a 12.5% solution of sodium hypochlorite was added to 530 grams of water. The sodium hypochlorite solution was mixed with the water followed by the addition of 240 grams of 50% sodium hydroxide solution at a rate to keep the temperature of the reaction below 30° C. After the addition of all the sodium hydroxide was completed, this mixture can be stored for later use or can be used for further reactions.

When further reactions were desired 66.7 grams of a 45% solution of ferric chloride was added to the hypochlorite—sodium hydroxide mixture in a reaction tank. The reaction was stirred and continuously monitored by UV-VIS for the production of ferrate and separately monitored to determine when there was no residual hypochlorite. The reaction took approximately 90 minutes with a yield of 25,000 ppm of ferrate measured as potassium ferrate. After the sodium ferrate was produced the mixture was diluted 9 to 1 with water and placed in the storage tank. At this point phosphate can be added for storage stability.

The process as shown in FIG. 5 was used to produce 50 gallons of the ferrate solution.

### Example 3

### Phosphate Stabilization

Addition of phosphate will stabilize the ferrate solution held in the storage tank to allow the solution to be held for extended holding periods. The addition of a 1:4 mixture of 0.05 M KH2PO4 to 0.05 M K2HPO4 was added to the diluted ferrate solution from Example 2. Stabilization was also obtained with a 1:19 mixture of the same components. The results indicated that with phosphate added the pH of the storage solution was no longer critical which gives more latitude for production and/or storage. For those cases where the pH of the system is required to be lower for a treatment application, phosphoric acid can be used to for stabilization and for addition to the treatment water.

The methods, procedures, and devices described herein are representative of preferred embodiments and are exemplary and are not intended as limitations on the scope of the invention. Substitutions and modifications may be made to the invention disclosed herein without departing from the scope and spirit of the invention.

Each and every patent and publication cited in this specification is individually incorporated by reference herein to the same extent as if each individual publication was specifically, separately and individually indicated to be incorporated by reference.

We claim:

1. A composition containing a solution comprising a first unstable oxidant, which is a product of a reaction comprising a second unstable oxidant reactant; wherein said composition is substantially free of said second unstable oxidant.

2. The composition of claim 1 wherein said first unstable oxidant comprises a metal ferrate component.

3. The composition of claim 1 or 2 wherein said second unstable oxidant comprises a hypochlorite component.

4. The composition of any of claims 1-3 wherein said first unstable oxidant has a half life in said composition of greater than about 25 minutes.

5. The composition of any of the foregoing claims wherein the first unstable oxidant is selected from the group consisting of potassium ferrate and sodium ferrate.

6. The composition of any of the foregoing claims wherein the second unstable oxidant is selected from the group consisting of a potassium hypochlorite component, a calcium hypochlorite component and a sodium hypochlorite component.

7. The composition of any of the foregoing claims wherein the second unstable oxidant is in a reaction solution comprising about 10% to about 13% of a metallic hydroxide; and having a pH of at least 12.

8. The composition of claim 7 wherein the metallic hydroxide is selected from the group comprising a sodium hydroxide and potassium hydroxide.

9. The composition of any of the foregoing claims wherein a stabilizing agent is not added to the composition to inhibit degradation of the first unstable oxidant.

10. The composition of any of claims 1-8 wherein a stabilizing agent is added to the composition to inhibit degradation of the first unstable oxidant.

11. The composition of any of the foregoing claims wherein the composition has a concentration of at least about 2% (w/v).

12. The composition of any of the foregoing claims wherein the composition has a concentration of at least about 1000 mg/l.

13. The composition of any of the foregoing claims wherein the composition has a concentration of at least about 10 mg/ml.

14. The composition of any of the foregoing claims wherein the composition has a concentration of at least about 8.5 mg/ml.

15. A method of treating materials with a biocide comprising contacting said materials with the composition of any of claims 1-14, wherein the unstable oxidant product comprises a ferrate and is stored at a concentration of at least about 1000 mg/ml and subsequently diluted and delivered to said materials at a substantially uniform concentration of at least about 8.5 mg/l sodium ferrate or at least about 10 mg/l potassium ferrate or any other metal ferrate, and wherein said composition lacks detectable hypochlorite, as monitored using spectrophotometry or ORP/pH.

16. A method for accurately producing a desired concentration of an unstable chemical product from an unstable chemical reactant at a substantially uniform rate over time comprising the steps of:
a). separately storing two or more reactants, at least one of which is unstable;
b) monitoring the concentration of the unstable reactant;
c) placing the stored reactants into each of at least two reaction vessels at different times, thereby initiating chemical reactions in each reaction vessel at different times;
d) monitoring the concentration of at last one reactant and at least one product in each reaction vessel until a first desired amount of an unstable product is produced free or substantially free of said unstable reactant, as monitored using spectrophotometry or ORP/pH measurement.

17. The method of claim 16 wherein the unstable product is a metal ferrate, further comprising:
a) transferring the metal ferrate from a reaction vessel into a storage or delivery vessel at a concentration of at least about 1000 mg/l,
b) monitoring the concentration of said metal ferrate continuously, or substantially continuously until the product reaches the site of use, and
c) accurately delivering at least about 8.5 mg/l sodium ferrate or at least about 10 mg/l potassium or other metal ferrates to said intended use.

18. The method of claim 16 further comprising adjusting the flow rate of the delivery stream from the two or more reaction vessels, to ensure substantially continuous delivery of the desired amount of said unstable product free or substantially free of said unstable reactant.

19. The method of any of claims 16-18 wherein, when the reaction is complete in one reaction vessel, and the unstable product is delivered therefrom, the vessel is refilled with reactants and a new reaction is initiated, thereby permitting said continuous delivery to be substantially uninterrupted.

20. The method of any of claims 16 and 18-19 wherein said unstable product is a metal ferrate.

21. The method of any of claims 16-20 in which said unstable reactant is a hypochlorite.

22. A method of synthesizing an unstable chemical solution of known concentration, comprising:
a) storing two or more reactants, at least one of which comprises an unstable oxidant component, in separate storage containers;
b) placing said reactants into at least two reaction vessels, monitoring the concentration of at least one unstable reactant and at least one unstable product, initiating identical chemical reactions in each reaction vessel at different times calculated to permit a substantially uninterrupted, substantially constant amount of the product to be synthesized, and;
c) collecting a volume of the unstable product substantially free of the unstable oxidant component.

23. The method of claim 22 wherein the unstable product is a metal ferrate.

24. The methods of claims 22-23 wherein the unstable oxidant component is a soluble hypochlorite.

25. A method of producing a substantially uncontaminated ferrate solution of a defined concentration using a hypochlorite reactant, comprising:
a) monitoring the concentration of a metal hypochlorite reactant prior to reaction;
b) placing reactants comprising a ferric halide, an alkali metal hydroxide and said hypochlorite into a reaction vessel in which the concentration of the hypochlorite reactant and a ferrate product are monitored, and initiating a chemical reaction;
c) removing the ferrate solution from the reaction vessel when the hypochlorite has been essentially completely consumed.
